(19) United States
(12) Patent Application Publication
(10) Pub. No.: US 2025/0060352 A1
(43) Pub. Date: Feb. 20, 2025

(54) METHOD FOR ESTIMATING THE REAL-TIME GAS-OIL RATIO OF A FLOWING OIL WELL

(71) Applicant: Saudi Arabian Oil Company, Dhahran (SA)

(72) Inventors: Majed Shammari, Alkhobar (SA); Wisam Shaker, Dhahran (SA); Abdulhamied Alsousy, Dhahran (SA)

(73) Assignee: Saudi Arabian Oil Company, Dhahran (SA)

(21) Appl. No.: 18/449,323

(22) Filed: Aug. 14, 2023

(51) Int. Cl. G01N 33/28; E21B 47/06; E21B 47/10; G01F 1/34

(52) U.S. Cl. CPC G01N 33/2841; E21B 47/06; E21B 47/10; G01F 1/34

(57) ABSTRACT

A method for estimating the gas-oil ratio (GOR) of a flowing oil well in real-time includes installing a first pressure downhole monitoring system in a flowing oil well, and installing a second pressure downhole monitoring system in a flowing oil well such that a pressure gradient can be calculated between a pressure measured by the first pressure downhole monitoring system and a pressure measured by the second pressure downhole monitoring system. Then the pressure is measured at the well-head of the oil well, and previous production test results of the oil well are reviewed in order to collect a liquid flow rate from the measured well-head pressure. Finally, known correlations between liquid flow rates and pressure gradients at constant GOR values are used to determine the GOR of the well.

### FIELD

Embodiments disclosed herein generally relate to the field of wellbore and oil reservoir performance and testing. More specifically, the field relates to a method for determining the real-time gas-oil ratio (GOR) of flowing oil wells.

### TECHNICAL BACKGROUND

When flowing oil is brought to the surface from a subterranean reservoir, it is common for gas to come out of solution as the oil nears standard temperature and pressure conditions. The gas-oil ratio (GOR) is a measure of the ratio of the volume of gas that comes out of the oil solution and the free gas phase in the reservoir compared to the volume of oil at these conditions.

Current methods of calculating the GORs of oil wells are not accurate due to the lack of regular calibration of the gas metering devices, and/or due the range limitations of these devices, especially in mature oil fields. Unreliable GOR measurements obtained from currently used methods affect the monitoring and controlling of gas production at the oil-producer level. For instance, an overestimation of a producer's GOR can lead to unnecessary work being done on an otherwise perfectly-fine well, which could possibly affect the region's sweep efficiency in a negative manner. On the other hand, an underestimation of a producer's GOR could lead to a missed opportunity to workover and recomplete the well.

As a result of the inaccurate GOR estimations of gas metering devices, portable testing separators are often used to confirm the measurements of these devices. However, relying on portable testing separators requires enormous planning, manpower, time, and large budgets. In mature oil fields, these requirements are exacerbated, as intensive testing is needed.

### SUMMARY

Accordingly, there is an ongoing need for an efficient and effective method for estimating the GOR of flowing oil wells.

According to one or more embodiments of the present disclosure, a method for estimating the gas-oil ratio (GOR) of a flowing oil well in real-time comprises: installing a first pressure downhole monitoring system in a flowing oil well; installing a second pressure downhole monitoring system in a flowing oil well such that a pressure gradient can be calculated between a pressure measured by the first pressure downhole monitoring system and a pressure measured by the second pressure downhole monitoring system; measuring the pressure at the well-head of the oil well; reviewing previous production test results of the oil well in order to collect a liquid flow rate from the measured well-head pressure; and using known correlations between liquid flow rates and pressure gradients at constant GOR values to determine the GOR of the well.

Additional features and advantages will be set forth in the detailed description which follows, and in part will be readily apparent to those skilled in the art from that description or recognized by practicing the embodiments described herein, including the detailed description which follows, the claims, as well as the appended drawings.

It is to be understood that both the preceding general description and the following detailed description describe various embodiments and are intended to provide an overview or framework for understanding the nature and character of the claimed subject matter. Additional features and advantages of the embodiments will be set forth in the detailed description, and, in part, will be readily apparent to persons of ordinary skill in the art from that description, which includes the accompanying drawings and claims, or recognized by practicing the described embodiments. The drawings are included to provide a further understanding of the embodiments, and together with the detailed description, serve to explain the principles and operations of the claimed subject matter. However, the embodiments depicted in the drawings are illustrative and exemplary in nature, and not intended to limit the claimed subject matter.

### BRIEF DESCRIPTION OF DRAWINGS

The following detailed description may be better understood when read in conjunction with the following drawings, in which:

FIG. 1 is a block flow diagram illustrating the steps of the method to estimate the real-time GOR of a flowing oil well;

FIG. 2 is a graph of the pressure gradient versus the liquid rate for estimating GORs in the range 550 to 1500;

FIG. 3 is a graph of the pressure gradient versus the liquid rate for estimating GORs in the range 1750 to 8000;

FIG. 4 is a graph displaying the pressure gradient versus the liquid rate for estimating the GORs in the range 500 to 8000; and

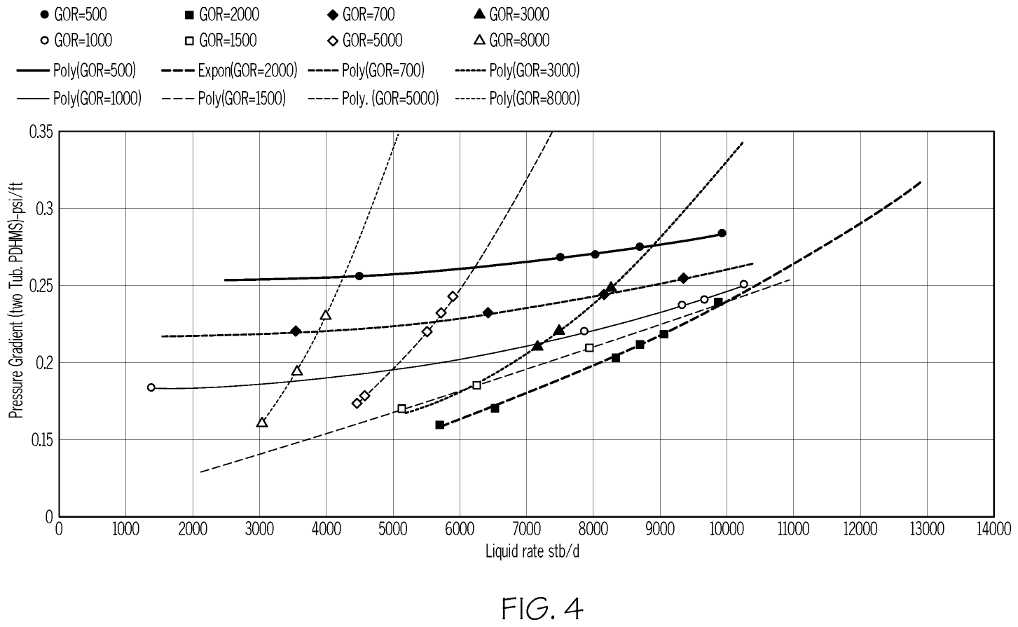

FIG. 5 is a graph of the general relationship of well head pressure (WHP) compared to liquid rate.

### DETAILED DESCRIPTION

Embodiments of the present disclosure are directed to processes for estimating the real-time GOR of a flowing oil well, and various embodiments are discussed herein. However, it should be understood that the forgoing detailed description section describes one or more specific embodiments and should not be viewed as limiting the scope of the appended claims.

As used throughout this disclosure, a “multiphase single-well model” refers to the process by which correlations between the liquid flow rate of an oil producer and a pressure gradient of the flowing oil within the producer at a constant GOR value are used to create correlation charts for each oil producer. The “multiphase single-well model” can be used to recalibrate the correlations for each oil producer when the productivity of the well has changed.

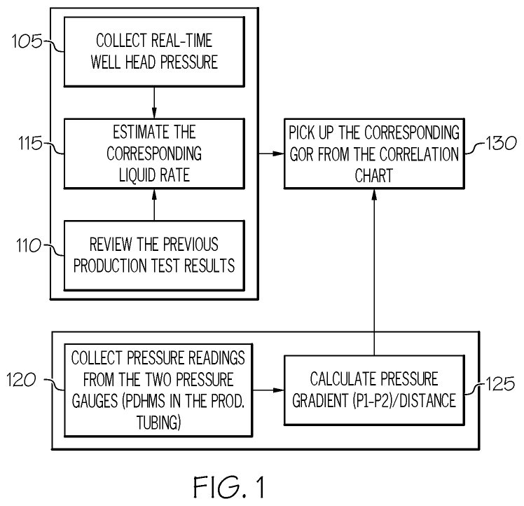

Now referring to FIG. 1, an embodiment of a method 100 for estimating the real-time GOR of a flowing oil well is depicted as a block flow diagram. The overall method 100 generally involves two steps: collecting the current liquid flow rate of the flowing oil well and calculating a pressure gradient within the same oil well. Once these two steps are complete, the liquid flow rate and the pressure gradient can be used to find the GOR from correlation charts. Previous production test results of flowing wells are reviewed 110 to find the correlation between well-head pressures and liquid rate. The real-time pressure at the well head 105 of the oil well is measured and compared with the previous production test results so that the estimate of the current liquid rate of the well can be determined. It should be understood that the real-time pressure at the well head may be measured before or after the previous production test results are reviewed according to embodiments disclosed and described herein. The pressure at the well-head may be measured by any suitable device. As described below, the liquid rate of the well can then be used with the pressure gradient to determine the GOR of the well.

In embodiments, the liquid rate is generally between 1000 stock tank barrels per day (stb/d) and 15000 stb/d. In embodiments, the pressure gradient is between 2000 stb/d and 5000 stb/d, 2000 stb/d and 6000 stb/d, 2000 stb/d and 7000 stb/d, or between 2000 stb/d and 8000 stb/d. In other embodiments, the calculated pressure gradient is between 1000 stb/d and 11000 stb/d. 3000 stb/d and 5000 stb/d, 4000 stb/d and 10000 stb/d, or between 5000 stb/d and 10000 stb/d.

In order to estimate the real-time GOR of the flowing oil, a pressure gradient of the oil well must also be calculated. In embodiments, this is accomplished by measuring the pressure from two separate pressure gauges 120 separated by a specified distance. Once the pressures are collected from the two pressure gauges 120, the pressure gradient of the flowing oil well can be calculated 125 using the following formula:

$$Pressure Gradient = \frac{P 1 - P 2}{D}$$

In the above formula used to calculate the pressure gradient of the flowing oil well, P1 is defined as the pressure of the flowing oil measured at gauge 1, and P2 is defined as the pressure measured at gauge 2. D is defined as the distance separating gauge 1 and gauge 2.

Accordingly, to measure the pressure gradient, a first pressure downhole monitoring system is installed in a flowing well, and a second pressure downhole monitoring system is installed in the flowing well a distance D from the first pressure downhole monitoring system. Thereby, a pressure gradient can be calculated-using the above equation-by measuring the pressure at the first pressure downhole monitoring system and measuring the pressure at the second pressure downhole monitoring system.

In embodiments, the well pressures are measured 120 using pressure downhole monitoring systems (PDHMS). As described above, embodiments of the currently disclosed method only require two PDHMSs. Further, in embodiments, the PHDMSs can be installed within a surface tubing of the oil well in order to function. In some embodiments, the PDHMSs are separated by a distance greater than 250 feet. In other embodiments, the PDHMSs are separated by a distance of less than 250 feet. In one or more embodiments, the PDHMSs are installed within the surface tubing proximal to the oil reservoir and are separated by a distance of 250 feet to 350 feet, such as 260 feet to 340 feet, 270 feet to 330 feet, 280 feet to 320 feet, or 290 feet to 310 feet. In a specific embodiment, the PDHMSs are separated by a distance of about 250 feet.

In embodiments, the pressure gradient is generally between 0.15 psi per foot (psi/ft) and 0.35 psi/ft. In other embodiments, the liquid flow rate is between 0.25 psi/ft and 0.30 psi/ft. 0.20 psi/ft and 0.30 psi/ft, or 0.15 psi/ft and 0.30 psi/ft. In further embodiments, the liquid flow rate is between 0.15 psi/ft and 0.25 psi/ft, or between 0.15 psi/ft and 0.20 psi/ft.

Once the pressure gradient has been calculated 125, and the corresponding liquid flow rate of the flowing oil well is collected from production testing database 115, the GOR of the well can be estimated 130 using the correlation charts for the relationship between the pressure gradient of the well and the liquid flow rate produced by the single-well multiphase model, as a strong correlation has been found between the pressure gradient and the liquid flow rate of a well at a constant GOR.

In embodiments, the estimated GOR for an oil producer is generally between 500 and 8000. In other embodiments, the estimated GOR is between 500 and 700, 500 and 1000, 500 and 1500, 500 and 2000, 500 and 2500, 500 and 3000, 500 and 4000, 500 and 6000, or between 500 and 7000. In certain embodiments, the estimated GOR is less than 4000, less than 3000, less than 2000, or less than 1500. A user may decide what GOR range is suitable for a specific reservoir based on past performance of the oil producer and general knowledge.

For each oil producer, a multiphase single-well model can be prepared to validate the correlation estimates for GOR values against real production testing results at various stages of depletion. From these results, it was found that the estimated GOR values by the correlation between the liquid flow rate of the well and the pressure gradient of the well were in high agreement with field measurements of the GOR measured via conventional portable production testing separators. For each oil producer, a correlation chart between the liquid flow rate of the producer and the measured pressure gradient must be prepared. Further, the correlation charts for each oil producer need to be updated when the productivity of the well is changed.

In further embodiments, the previous production test results are reviewed 110 in order to accurately estimate the liquid flow rate of the well. In embodiments, the oil well producers are tested at three chokes on a monthly or quarterly basis. From the production testing at the various chokes, a well behavior plot is produced, which provides a relationship between the liquid flow rate and well head pressures. The well behavior plots then allow for an accurate collection of the current liquid flow rate of the well at various measured well head pressures.

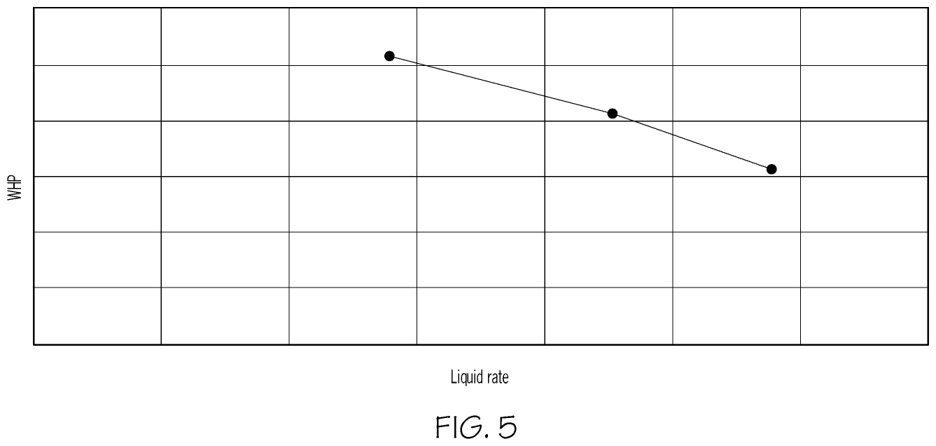

With reference to FIG. 5, the general relationship between well head pressure (WHP) and liquid rate may be characterized as a decline, as in WHP decreases as the liquid rate increases. The slope of WHP remains linear for a time until it declines more rapidly after a certain point. This general relationship may vary depending upon user productivity and selection of appropriate GOR range.

In view of the above, aspects for estimating the GOR of a flowing oil well include:
Aspect 1 is a method for estimating the gas-oil ratio (GOR) of a flowing oil well in real-time, the method comprising: installing a first pressure downhole monitoring system in a flowing oil well; installing a second pressure downhole monitoring system in a flowing oil well such that a pressure gradient can be calculated between a pressure measured by the first pressure downhole monitoring system and a pressure measured by the second pressure downhole monitoring system; measuring the pressure at the well-head of the oil well; reviewing previous production test results of the oil well in order to collect a liquid flow rate from the measured well-head pressure; and using known correlations between liquid flow rates and pressure gradients at constant GOR values to determine the GOR of the well.
Aspect 2 is the method of aspect 1, wherein the GOR correlations between liquid flow rates and pressure gradients are calculated using a calibrated multiphase single-well model.
Aspect 3 is the method of any preceding aspect, wherein the GOR correlations estimated by the multiphase single-well model are validated via a portable production testing separator.
Aspect 4 is the method of any preceding aspect, wherein the pressure downhole monitoring systems are separated by a distance of between 250 and 350 feet.
Aspect 5 is the method of any preceding aspect, wherein the pressure downhole monitoring systems are installed within the surface tubing of the oil well.
Aspect 6 is the method of any preceding aspect, wherein the pressure downhole monitoring systems are installed proximal to the oil reservoir.
Aspect 7 is the method of any preceding aspect, wherein the estimated GOR is less than 8000.
Aspect 8 is the method of any preceding aspect, wherein the estimated GOR is less than 1500.
Aspect 9 is the method of any preceding aspect, wherein the GOR is estimated with greater than 80% accuracy of the measured GOR when the GOR is less than 1500.
Aspect 10 is the method of any of aspects 1 through 7, wherein the GOR is estimated with greater than 70% accuracy of the measured GOR when the GOR is greater than 1500.
Aspect 11 is the method of any preceding aspect, wherein the calculated pressure gradient is between 0.15 psi per foot (psi/ft) and 0.35 psi per foot (psi/ft).
Aspect 12 is the method of any preceding aspect, wherein the measured liquid flow rate is between 1000 stock tank barrels per day (stb/d) and 15000 stock tank barrels per day (stb/d).

### EXAMPLES

Embodiments will be further clarified by the following examples.

### Example 1

Referring to FIG. 1, an example plot describing the robust correlation between the estimated liquid flow rate of the well and the pressure gradient is depicted. In generating the plot, multiphase well modelling was used to predict the pressure gradient at the PDHMSs against different assumed liquid flow rates at specific GORs. This procedure was repeated to cover a selected GOR range. In FIG. 1, the correlations between the liquid flow rate and the pressure gradient are shown for the GOR range of 550 to 1500. The markers on the graph refer to specific data points of a pressure gradient versus the liquid flow rate. The various trend lines were then fit to each set of data corresponding to a specific GOR. Thus, given a measured pressure gradient from the PDHMSs and an estimated liquid flow rate, the GOR of the oil producer can be estimated in real-time.

TABLE 1

| | Estimated | Measured GOR via | |
|---|---|---|---|
| Well | GOR Using | portable production | % |
| Number | Multiphase Model | testing separators | Accuracy |
| 1 | 550 | 650 | 84.6% |
| 2 | 550 | 670 | 82.1% |
| 3 | 550 | 530 | 96.4% |
| 4 | 700 | 604 | 86.2% |
| 5 | 1000 | 1004 | 99.6% |

In Table 1 above, the GOR results for 5 oil producers are shown. For each well 1-5, the GOR of the well was estimated using the single-well multiphase model described above. Additionally, in each well 1-5, conventional portable production testing separators were utilized to measure the actual GOR of the well. The agreement between the GOR value predicted by the multiphase model and the GOR value measured via the portable production testing separators is displayed in the Accuracy column. The values in the accuracy column are calculated by dividing the difference between the predicted GOR and the measured GOR by the larger of the two values. As can be seen, the GOR values estimated using the multiphase model in wells 1-5 agree with the measured values well, allowing for robust estimation of the GOR for an oil producer using the presently disclosed method.

### Example 2

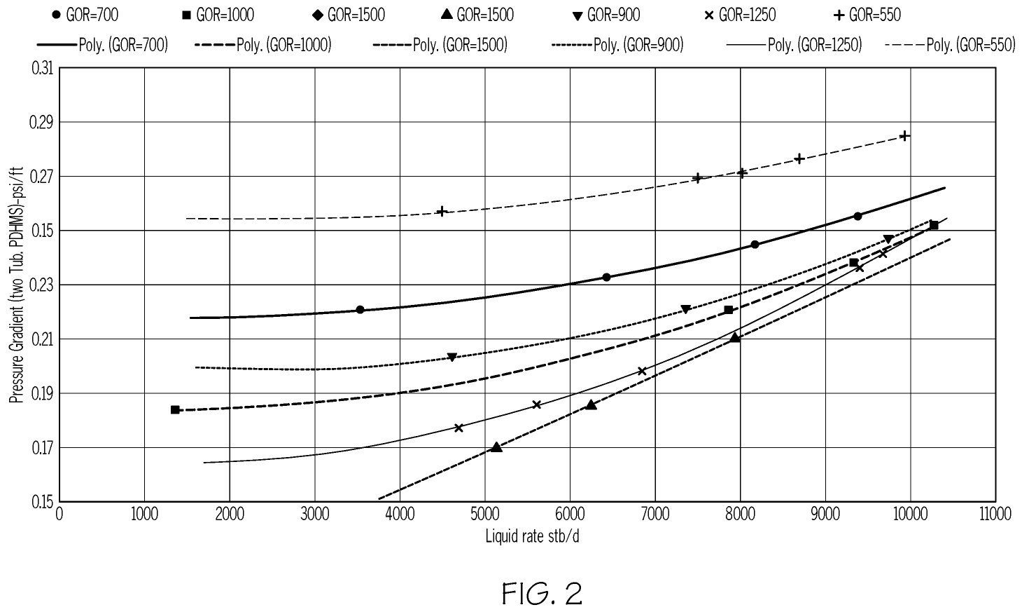

Referring to FIG. 2, another example plot describing the correlation between the estimated liquid flow rate of the producer and the pressure gradient is depicted. The plot is generated using the same procedure described above in Example 1, where multiphase well modelling was used to predict the pressure gradient at the PDHMSs against various assumed liquid flow rates at specific GORs. This plot, however, displays the correlation between a different range of GOR values. Specifically, the range of GOR correlations depicted is between 1750 and 8000. The markers again refer to specific data points of various pressure gradients plotted against an estimated liquid flow rate, and trend lines are then fit to the data points, producing a trend line for each GOR value.

TABLE 2

| | Estimated | Measured GOR via | |
|---|---|---|---|
| Well | GOR Using | portable production | % |
| Number | Multiphase Model | testing separators | Accuracy |
| 6 | 2000 | 1500 | 75.0% |
| 7 | 2000 | 2300 | 86.9% |
| 8 | 4000 | 3122 | 78.1% |
| 9 | 6000 | 7084 | 84.7% |

In Table 2 above, the GOR results for four other wells, 6-9, is shown. Again, for each well, the GOR was estimated using the multiphase model described above. Further, portable production testing separators were used to test the agreement between the estimated GOR values and measured GOR values for each oil producer. The values in the Accuracy column reflect the agreement between the values for the estimated GOR using the multiphase model and the measured GOR via the portable production testing separators. As can be seen, there is less agreement between the two GOR values as the GOR values become large. Without being bound by any particular theory, it is believed that at high GOR values, the portable phase separators fail to measure the GOR values effectively.

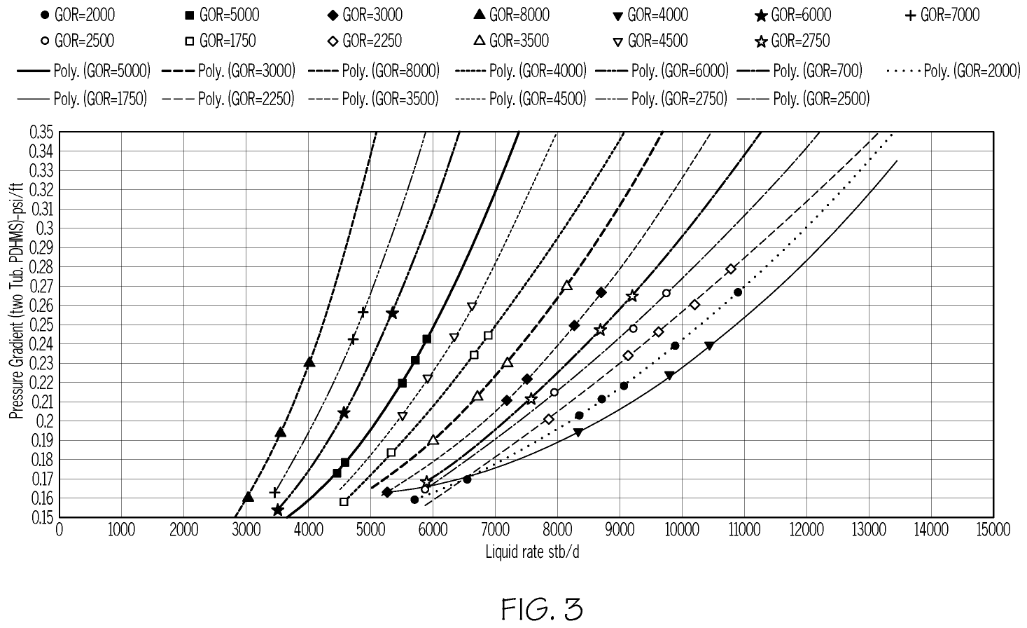

Referring to FIG. 3, a third example of a correlation plot for estimating the GOR of an oil producer is depicted. As with FIG. 1 and FIG. 2, the plot is generated using the single-well multiphase model. For this oil well, the correlations for estimating a GOR between 500 and 8000 are depicted. The markers on the graph refer to specific data points of a pressure gradient versus the liquid flow rate. The various trend lines were then fit to each set of data corresponding to a specific GOR.

It will be apparent to persons of ordinary skill in the art that various modifications and variations can be made without departing from the scope disclosed herein. Since modifications, combinations, sub-combinations, and variations of the disclosed embodiments, which incorporate the spirit and substance disclosed herein, may occur to persons of ordinary skill in the art, the scope disclosed herein should be construed to include everything within the scope of the appended claims and their equivalents.

For the purposes of defining the present technology, the transitional phrase “consisting of” may be introduced in the claims as a closed preamble term limiting the scope of the claims to the recited components or steps and any naturally occurring impurities. For the purposes of defining the present technology, the transitional phrase “consisting essentially of” may be introduced in the claims to limit the scope of one or more claims to the recited elements, components, materials, or method steps as well as any non-recited elements, components, materials, or method steps that do not materially affect the novel characteristics of the claimed subject matter. The transitional phrases “consisting of” and “consisting essentially of” may be interpreted to be subsets of the open-ended transitional phrases, such as “comprising” and “including,” such that any use of an open ended phrase to introduce a recitation of a series of elements, components, materials, or steps should be interpreted to also disclose recitation of the series of elements, components, materials, or steps using the closed terms “consisting of” and “consisting essentially of.” For example, the recitation of a composition “comprising” components A, B, and C should be interpreted as also disclosing a composition “consisting of” components A, B, and C as well as a composition “consisting essentially of” components A, B, and C. Any quantitative value expressed in the present application may be considered to include open-ended embodiments consistent with the transitional phrases “comprising” or “including” as well as closed or partially closed embodiments consistent with the transitional phrases “consisting of” and “consisting essentially of.”

As used in the Specification and appended Claims, the singular forms “a”, “an”, and “the” include plural references unless the context clearly indicates otherwise. The verb “comprises” and its conjugated forms should be interpreted as referring to elements, components or steps in a non-exclusive manner. The referenced elements, components or steps may be present, utilized or combined with other elements, components or steps not expressly referenced.

It should be understood that any two quantitative values assigned to a property may constitute a range of that property, and all combinations of ranges formed from all stated quantitative values of a given property are contemplated in this disclosure. The subject matter disclosed herein has been described in detail and by reference to specific embodiments. It should be understood that any detailed description of a component or feature of an embodiment does not necessarily imply that the component or feature is essential to the particular embodiment or to any other embodiment. Further, it should be apparent to those skilled in the art that various modifications and variations can be made to the described embodiments without departing from the spirit and scope of the claimed subject matter.

1. A method for estimating the gas-oil ratio (GOR) of a flowing oil well in real-time, the method comprising:
installing a first pressure downhole monitoring system in a flowing oil well;
installing a second pressure downhole monitoring system in a flowing oil well such that a pressure gradient can be calculated between a pressure measured by the first pressure downhole monitoring system and a pressure measured by the second pressure downhole monitoring system;
measuring the pressure at a well-head of the oil well;
reviewing previous production test results of the oil well and collecting a liquid flow rate from the measured well-head pressure; and
using known correlations between liquid flow rates and pressure gradients at constant GOR values to determine the GOR of the oil well.

2. The method of claim 1, wherein the known correlations between liquid flow rates and pressure gradients are calculated using a calibrated multiphase single-well model.

3. The method of claim 1, wherein the known correlations estimated by the multiphase single-well model are validated via a portable production testing separator.

4. The method of claim 1, wherein the first pressure downhole monitoring system and the second pressure downhole monitoring system are separated by a distance of between 250 feet and 350 feet.

5. The method of claim 4, wherein the first pressure downhole monitoring and the second pressure downhole monitoring system are installed within a surface tubing of the oil well.

6. The method of claim 4, wherein the first pressure downhole monitoring and the second pressure downhole monitoring system are installed proximal to an oil reservoir.

7. The method of claim 1, wherein the estimated GOR is less than 1500.

8. The method of claim 1, wherein the estimated GOR is less than 8000.

9. The method of claim 1, wherein the GOR is estimated with greater than 80% accuracy of the measured GOR when the GOR is less than 1500.

10. The method of claim 1, wherein the GOR is estimated with greater than 70% accuracy of the measured GOR when the GOR is greater than 1500.

11. The method of claim 1, wherein the calculated pressure gradient is between 0.15 psi per foot (psi/ft) and 0.35 psi per foot (psi/ft).

12. The method of claim 1, wherein the measured liquid flow rate is between 1000 stock tank barrels per day (stb/d) and 15000 stock tank barrels per day (stb/d).

13. The method of claim 5, wherein the pressure downhole monitoring systems are installed proximal to the oil reservoir.
